(19) United States
(12) Patent Application Publication
(10) Pub. No.: US 2008/0108974 A1
(43) Pub. Date: May 8, 2008

(54) Reinforced catheter with radiopaque distal tip and process of manufacture

(75) Inventor: Fung May Yee Roth, Denver, PA (US)

Correspondence Address: R. GALE RHODES, ESQ. / MOSER IP LAW GROUP, 1030 BROAD STREET, 2ND FLOOR, SHREWSBURY, NJ, 07702, US

(73) Assignee: Vital Signs, Inc.

(21) Appl. No.: 11/584,703

(22) Filed: Oct. 20, 2006

(51) Int. Cl. A61M 25/098

(52) U.S. Cl. 604/529

(57) ABSTRACT

A catheter, including: a reinforced tube including a plurality of concentric bonded tubular layers of non-radiopaque material and a radiopaque, coil spring captured between adjacent ones of the tubular layers, one end of the reinforced tube providing an annular mounting portion and a tubular mounting member extending outwardly of the annular mounting portion; and a tapered tip of radiopaque material including a tip central portion providing a tip central passageway receiving the tubular mounting member and a tip mounting portion abutting and bonded to the annular mounting portion, the tip central portion bonded to the tubular mounting member. The process of manufacturing a catheter, including the steps of: providing a reinforced tube including a plurality of concentric bonded tubular layers of non-radiopaque material and a radiopaque, coil spring captured between adjacent ones of the tubular layers, providing an annular mounting portion at one end of the reinforced tube and providing a tubular mounting member extending outwardly of the annular mounting portion, and providing a tapered tip of radiopaque material including a tip central portion providing a tip central passageway and a tip annular mounting portion, bonding the tip annular mounting portion to the tube annular mounting portion and bonding the tip central portion to the tubular mounting member.

### BACKGROUND OF THE INVENTION

The present invention relates generally to a catheter. More particularly the present invention relates to a reinforced catheter having a radiopaque distal tip, and still more particularly, relates to kink-resistant, flat-wire reinforced catheter having a soft radiopaque distal tip.

Percutaneous interventional procedures often require the use of catheters to negotiate, i.e. pass through, arteries, veins or interstitial spaces from the site of entry into the body to the site requiring treatment or study. The catheter may either provide a conduit for delivery of therapeutic devices like angioplasty systems, stent delivery systems, pacing leads, guide wires, biopsy devices or intravascular ultrasound devices; or provide a mode for drug or fluid delivery. Introducer sheaths or introducer catheters or guiding catheters, for instance, are of this type. In other instances, the catheter may be part of the therapeutic device, e.g., some of the aforementioned.

Such catheters are often required to be highly flexible, kink resistant, pushable and of minimal wall thickness. The distal tips of such catheters are often required to track a guide wire while minimizing trauma within the body. In addition, physicians often expect the distal tip as well as any other portion of the catheter that is placed in the body, to be identifiable under fluoroscopy (radiopaque) enabling visual feedback during the positioning and use of the catheter.

The present invention provides a catheter that is highly flexible, kink-resistant, pushable, of minimal wall thickness, reinforced with a coil spring of radiopaque material, and tracks a guide wire with a soft, atraumatic, radiopaque distal tip.

### SUMMARY OF THE INVENTION

A catheter, including: a reinforced tube including a plurality of concentric bonded tubular layers of non-radiopaque material and a radiopaque, coil spring captured between adjacent ones of the tubular layers, one end of the reinforced tube providing an annular mounting portion and a tubular mounting member extending outwardly of the annular mounting portion; and a tapered tip of radiopaque material including a tip central portion providing a tip central passageway receiving the tubular mounting member and a tip mounting portion abutting and bonded to the annular mounting portion, the tip central portion bonded to the tubular mounting member.

The process of manufacturing a catheter, including the steps of providing a reinforced tube including a plurality of concentric bonded tubular layers of non-radiopaque material and a radiopaque, coil spring captured between adjacent ones of the tubular layers, providing an annular mounting portion at one end of the reinforced tube and providing a tubular mounting member extending outwardly of the annular mounting portion, and providing a tapered tip of radiopaque material including a tip central portion providing a tip central passageway and a tip mounting portion, bonding the tip mounting portion to the annular mounting portion and bonding the tip central portion to the tubular mounting member.

### BRIEF DESCRIPTION OF THE DRAWINGS

An understanding of the invention may be had by reference to embodiments of the invention illustrated in the appended drawings. It is to be noted, however, that the appended drawings illustrate only typical embodiments of this invention and are therefore not to be considered limiting of its scope, for the invention may admit to other equally effective embodiments.

FIG. 1 is an outline view of the first embodiment of a catheter embodying the present invention;

FIG. 2 is a longitudinal cross-sectional view of the catheter of FIG. 1;

FIG. 3 is an exploded view showing the distal end of the reinforced tubing of the catheter in cross-section and showing a tapered radiopaque distal tip in perspective;

FIG. 4 is a right side, end view of the distal portion of the reinforced tubing shown in FIG. 3 without the distal tip.

FIGS. 5-10 illustrate a process for manufacturing the catheter shown in FIGS. 1-4;

FIG. 11 is an outline view of a further embodiment of a catheter embodying the present invention;

FIG. 12 Is a longitudinal cross-sectional view of the catheter shown in FIG. 11;

FIG. 13 is an exploded view showing the distal portion of the reinforced tubing in cross-section comprising the catheter embodiment shown in FIG. 11 and showing the tapered radiopaque distal tip in perspective; and

FIG. 14 is a right side, end view of the reinforced tubing shown in cross-section in FIG. 13 without the distal tip.

### DETAILED DESCRIPTION

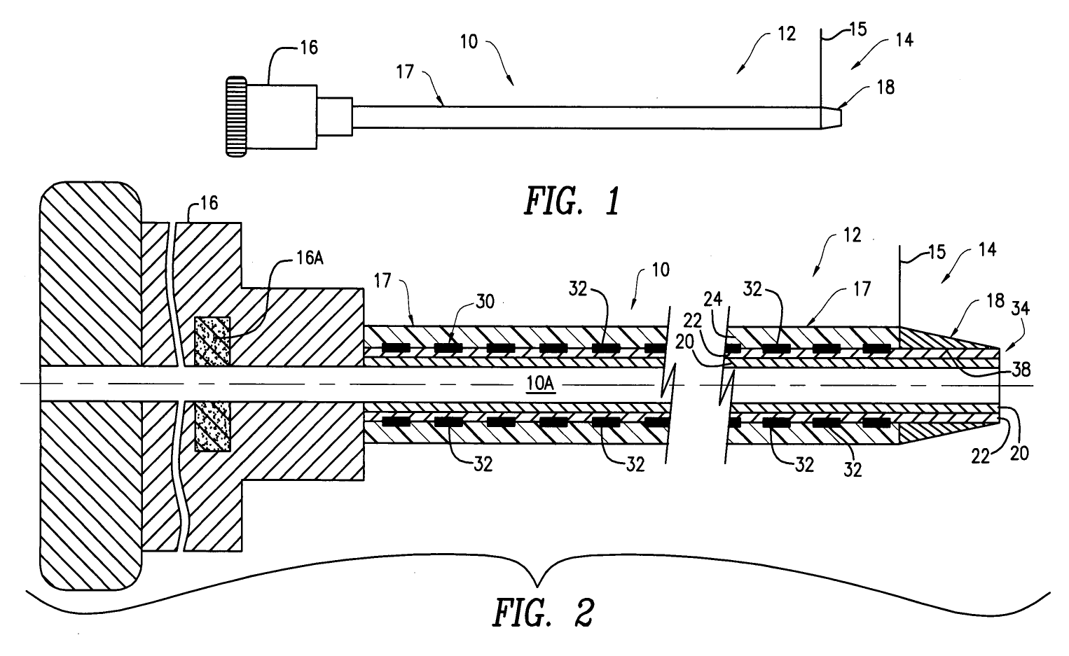

A first embodiment of a catheter embodying the present invention is illustrated in FIGS. 1 and 2 and is identified by general numerical designation 10; the catheter is provided with a central catheter passageway 10A. The catheter 10 includes a proximal portion indicated by general numerical designation 12 and a distal portion indicated by general numerical designation 14. The proximal portion Is to the left of the diagrammatical line 15 shown in FIGS. 1 and 2, and the distal portion is to the right of the vertical line 15. The vertical line 15 is for indicating, diagrammatically, the approximate demarcation between the proximal portion 12 and the distal portion 14 of the catheter 10. The proximal portion 12 may include a hub or adapter, indicated by general numerical designation 16 and a cylindrical proximal reinforced tube or tubing indicated by general numerical designation 17. The hub 16 may, or may not, include a hemostasis valve, indicated diagrammatically by numerical designation 16A, and which may be any one of several hemostasis valves known to the art for, by way of example, sealing around the outside surface of, such as for example, a guide wire when it is in place in the catheter passageway 10A to prevent loss of fluid or entry of air embolism. The distal portion of the catheter 10 includes a tapered or tapered radiopaque distal tip indicated by general numerical designation 18.

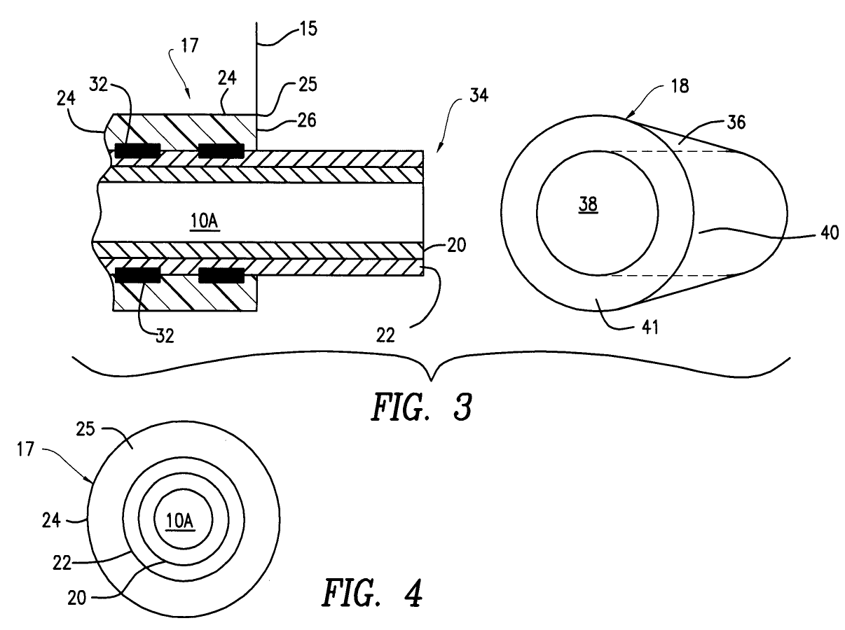

Referring to FIG. 2, the reinforced tube 17 includes a central tubular layer 20, of low friction, non-radiopaque material, extending through the catheter proximal portion 12 and through the catheter distal portion 14, as shown, and providing a catheter central passageway 10A An inner tubular layer 22, of non-radiopaque material, surrounds and is bonded to the inner tubular layer 20 and also extends through the catheter proximal catheter portion 12 and through the catheter distal portion 14 as shown. An outer tubular layer 24, of non-radiopaque material, surrounds and Is bonded to the inner tubular layer 22 and, as shown in FIG. 2, extends only through the catheter proximal portion 12. It will be understood from FIG. 3, that the outer tubular layer 24 includes a distal portion or annular tube mounting portion 25, providing, as shown in FIGS. 3 and 4, a tube annular mounting surface 26. The annular tube mounting portion 25 and the tube annular mounting surface 26, as described in detail below, are for mounting the distal tip 18 to the reinforced tube 17. As will be further understood from FIGS. 2 and 3, and as noted above, the central tubular layer 20 and the inner tubular layer 22 include portions extending into the catheter distal portion 14 and which portions, as will be particularly understood from FIG. 3, combine to provide a tubular mounting member indicated by general numerical designation 34. As described in detail below, the tubular mounting member 34 also is for mounting the distal tapered tip 18 to the reinforced tube 17.

The reinforced tube 17, FIG. 2, further includes a radiopaque, coil spring identified by general numerical designation 30 and which radiopaque, coil spring is indicated diagrammatically in FIG. 2 by the opposed rows of dark dashes 32; the dark dashes 32 also indicate, diagrammatically, the turns of the coil spring 30 which have spaces or gaps therebetween. Preferably, the coil spring is a flat-wire coil spring made from radiopaque, flat wire, such as for example, 304 stainless steel, about 0.003 inch to about 0.005 inch thick and which has a width that is less than about 4 times the thickness. As further indicated diagrammatically in FIG. 2, the radiopaque, coil spring 30 is captured between the outer tubular layer 24 and the inner tubular layer 22 with portions of the outer tubular layer 24 filling some of the gaps between the turns of the coil spring 30 and with portions of the inner tubular layer 22 filling other of the gaps. The coil spring 30 provides kink and crush resistance to the catheter 10, contributes to the flexibility of the catheter, facilitates a thin wall section for the catheter, and provides radiopacity for the proximal portion of the catheter.

The tapered distal tip 18 of radiopaque material, FIGS. 2 and 3, and particularly FIG. 3, includes a tip central portion 36 providing a tip central passageway 38 for receiving, as shown in FIG. 2, the tubular mounting member 34, and further includes a proximal portion, or tip mounting portion 40, providing a tip annular mounting surface 41. As shown in FIG. 2, and as described in detail below, the tip mounting portion 40 abuts and is bonded to the annular mounting portion 25 of the outer tubular layer 24, more particularly the tip annular mounting surface 41 (FIG. 3) abuts and is bonded to the annular mounting surface 26 (FIG. 3) of the outer tubular layer 24; the tip central portion 36 (FIG. 3) is bonded to the tubular mounting member 34 (FIG. 3) and, in particular, directly to the distal portion of the inner tubular layer 22.

Referring further to FIG. 2, the central tubular layer 20 may be a tubular layer of suitable low friction, non-radiopaque thermoplastic material, such as for example, fluoroethylene-propylene (FEP) or polytetrafluoroethylene (PTFE) which are low friction and non-radiopaque materials which will provide a lubricious conduit, e.g., catheter central passageway 10A, for medical devices of the type mentioned above passing through the catheter central passageway 10A. In the preferred embodiment, the central tubular layer 20 has a thickness of about 0.0005 inch to about 0.002 inch prior to fusing or bonding as described below.

The inner tubular layer 22 may be a suitable tubular layer of non-radiopaque thermoplastic material such as, for example, polyether block amide having a durometer of about 20 to about 30 on the Shore D scale. The outer tubular layer 24 may be a suitable tubular layer of non-radiopaque material such as polyether block amide having a durometer of about 50 to about 70 on the Shore D scale. In the preferred embodiment the inner tubular layer 22 has a thickness of about 0.001 inch to about 0.003 inch prior to fusing or bonding as described below. The wall thickness of the harder outer tubular layer 24 is dependent on the desired wall thickness and desired stiffness of the catheter, however in the preferred embodiment the outer tubular layer 24 had a thickness of about 0.0025 inch to about 0.005 inch prior to bonding as described below. The harder outer tubular layer 24 provides a smooth, non-tacky outer surface to the catheter 10 that is desirable for traversing the cardiovascular system or interstitial spaces. The softer inner tubular layer 22 is a tackler material than the harder outer layer 24 but of the same material family and thereby facilitates bonding to the harder outer layer 24. Still further, the polyether block amide of the outer tubular layer 24 may be compounded with light or processing stabilizers, or a colorant if desired.

The tapered distal tip 18 may be made of a suitable thermoplastic material filled with a suitable radiopaque agent such as, for example, polyether block amide having a durometer of about 30 to about 45 on the Shore D scale and which is filled with about 70% to about 90% by weight tungsten to make the tip radiopaque. This radiopaque material of the noted durometer provides a smooth, non-tacky surface that is desirable for traversing the cardiovascular system or interstitial spaces and further contributes to the flexible, atraumatic distal tip that facilitates tracking a guide wire.

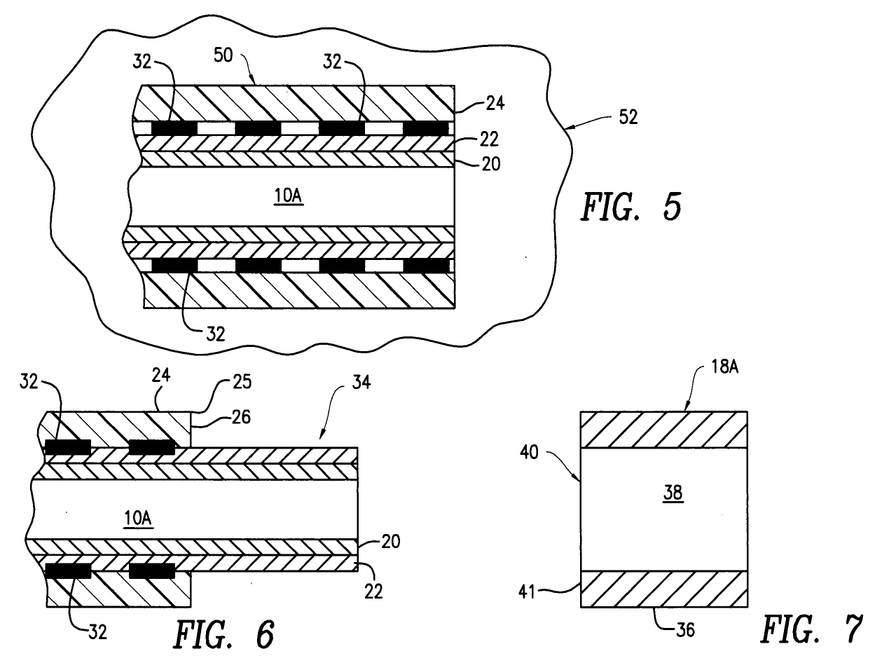

A process for manufacturing the catheter 10 is illustrated in connection with FIGS. 5-10. Referring to FIG. 5, the central tubular layer 20 is extruded over a cylindrical mandrel (not shown), the inner tubular layer 22 is extruded over the central tubular layer 20, the radiopaque, coil spring 30 is wound over the inner tubular layer 22 and the outer tubular layer 24 is extruded over the radiopaque, coil spring 30 and the inner tubular layer 22. The sub-assembly shown in FIG. 5 and which sub-assembly is indicated by general numerical designation 50 is then inserted in a suitable shrink tubing or jacket indicated diagrammatically by the surrounding irregular line balloon in FIG. 5 and indicated by general numerical designation 52; the shrink tubing may be, for example, fluoroethylene-propylene(FEP) shrink tubing. The shrink tubing wrapped sub-assembly 50 is suitably heated for a suitable period in the manner known to the art for bonding or fusing thermoplastic materials using heat shrink tubing. This heat shrink process step bonds the inner tubular layer 22 to the outer tubular layer 24 capturing the radiopaque, coil spring 30 between the inner tubular layer 22 and the outer tubular layer 24 as shown in FIG. 6 with portions of the inner tubular layer 22 filling some of the gaps or spaces between adjacent turns of the radiopaque, coil spring 30 and with portions of the outer tubular layer 24 filling other of such spaces or gaps. The sub-assembly 50 is cooled and the shrink tubing 52 removed. Then, the mandrel is removed.

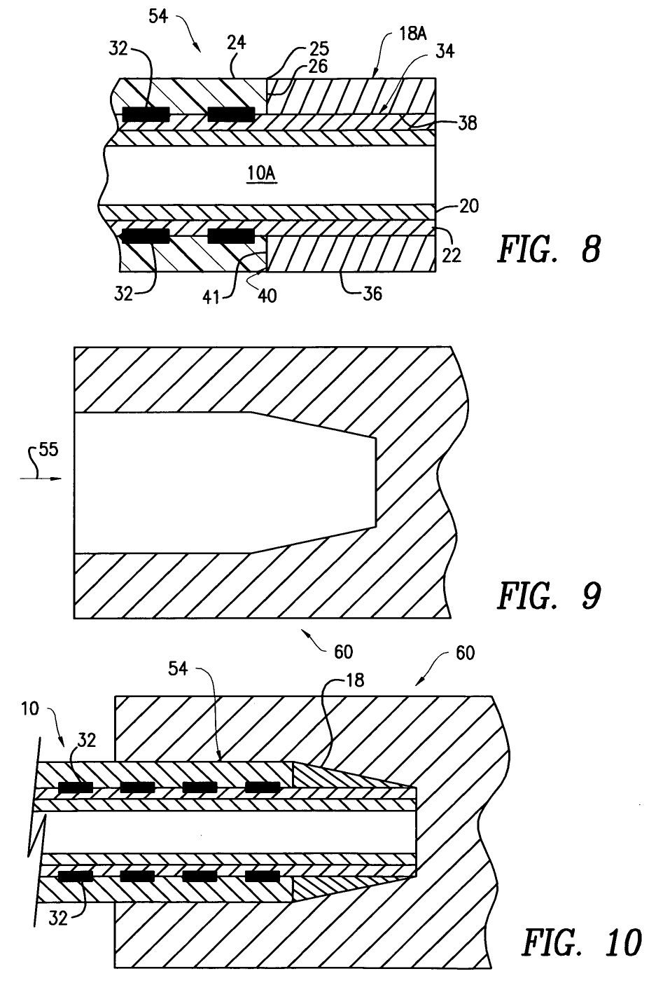

As shown in FIG. 6, the rightward end portions of the outer tubular layer 24 and the coil spring 30 are suitably removed such as by trimming away material to provide the tube mounting portion 25 and the annular mounting surface 26 and to expose the rightward end portions of the central tubular layer 20 and the inner tubular layer 22 to provide the tubular mounting member 34. A hollow cylindrical tube, or tubular layer, indicated by general numerical designation 18A, and shown in cross-section in FIG. 7, is provided of the material noted above for the tapered distal tip 18 and which tube 18A includes a central portion 36 providing the tip central passageway 38, the tip mounting portion 40 and the annular tip mounting surface 41. Thereafter, the cylindrical tube 18A is placed, or slides, over the tubular mounting member 34 to cause the mounting portion 40 of the tip 18A to abut the annular mounting portion 25 of the outer tubular layer 24, and more particularly, to cause the distal tip annular mounting surface 41 to engage the tube annular mounting surface 26, and to cause the distal tip central passageway 38 to receive the tubular mounting member 34 with the tip central portion 36 engaging the tubular mounting member 34, particularly the inner tubular member 22; this provides an assembly indicated by general numerical designation 54 in FIG. 8. The assembly 54 is inserted, as indicated by the arrow 55 in FIG. 9, into a heated tipping die, indicated by general numerical designation 60 in FIG. 9, to mold the rightward portion of the assembly 54 into the desired shape for the rightward end portion of the catheter 10 (FIG. 1), particularly into the desired end shape for the tapered distal tip 18. The tipping die 60 is heated to a temperature of about 150 C to about 210° C. to provide the tube 18A (FIG. 7) of the above-noted radiopaque material with the desired tapered shape of the tapered distal tip 18 as shown in FIG. 10, and to bond the distal tip annular mounting surface 41 (FIG. 3) to the tube annular mounting surface 26 (FIG. 3) and bond the distal tip central portion 36 (FIG. 7) to the tubular mounting member 34 (FIG. 6), particularly to the inner tubular layer 22 (FIG. 8). The rightward portion of the catheter 10 is removed from the tipping die 60 and cooled, and any required final trim operation is performed. It will be understood that the outer ends of the outer tubular layer 24 and the radiopaque, coil spring 30 can be prepared to provide the tubular mounting member 34 (FIG. 6) either before or after the heat shrink tubing process step described above. If the tubular mounting member 34 is prepared before, the tube 18A can also be added before the heat shrink tubing process.

In an alternate process of manufacturing the catheter 10, the tubular layers 20, 22 and 24 of FIG. 5 are provided as individual tubular layers and assembled as shown in FIG. 5, with the radiopaque, coil spring 30 wrapped around the inner tubular layer 22, to provide the sub-assembly 50. Thereafter the same process manufacturing steps described above in connection with FIGS. 5-10 are practiced or performed

Referring again to FIG. 2, the hub or adapter 16 is suitably formed into the shape shown such as being molded from a suitable thermoplastic material such as Isoplast or machined from a block of such thermoplastic material. The hub 16 is provided with the hemostasis valve 16A and is bonded or attached to the reinforced tube 17 by heat bonding or by a suitable adhesive known to the art for adhering plastic parts together.

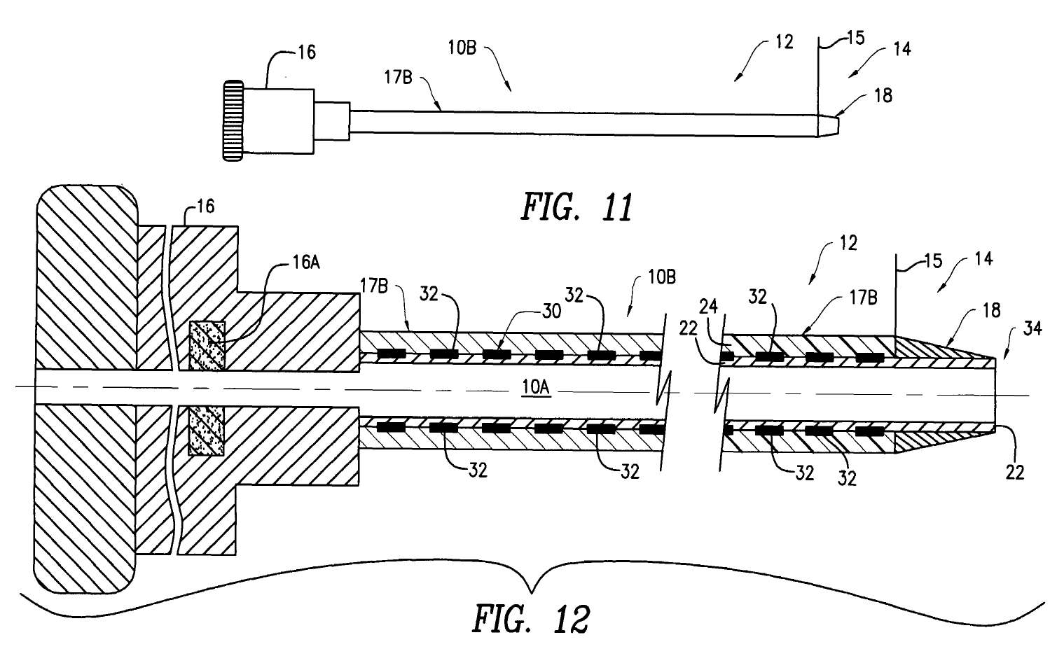

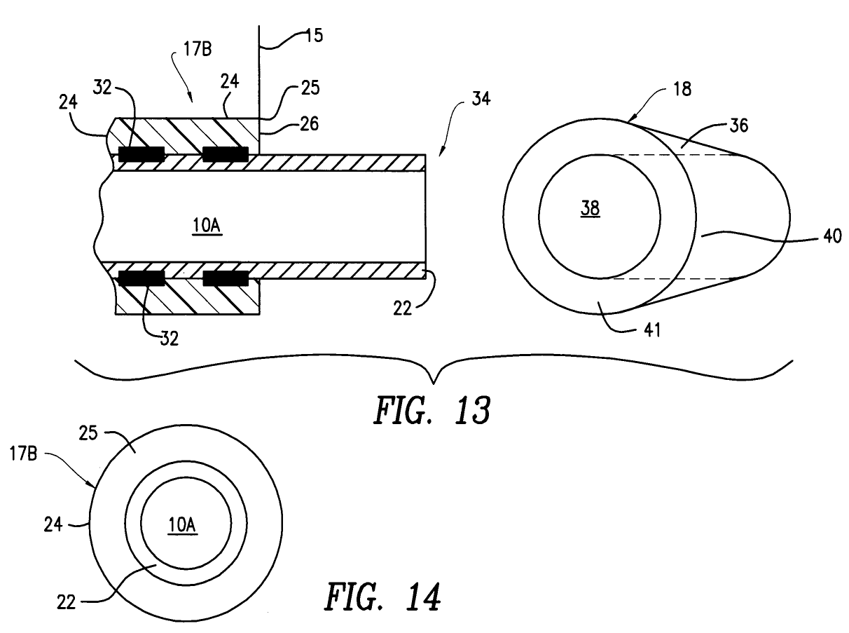

A further embodiment of a catheter embodying the present invention is illustrated in FIGS. 11-14 and indicated by general numerical designation 10B. Catheter 10B includes reinforced tube or tubing 17B, hub 16 and tapered distal tip 18 of radiopaque material. It will be understood that the elements or components comprising the catheter 10B which are the same as the elements or components comprising the catheter 10 shown in FIGS. 1-4 are given the same numbers in FIGS. 11-14 and will be understood to perform the same functions. Catheter 10B differs from catheter 10 in that the reinforced tube or tubing 17B does not include the inner tubular layer 22 of non-radiopaque material shown in FIGS. 24. Since the inner tubular layer 22 is not included in the reinforced tube or tubing 17B, it will be understood from FIG. 12 that the outwardly extending distal portion of the central tubular layer 20 provides the tubular mounting member 34 to which, the tapered distal tip 18 is bonded. It will be further understood that the process for manufacturing the catheter 10B is the same as the process described above for manufacturing the catheter 10 except that the inner tubular layer 22 of the catheter 10 is not incorporated in the manufacturing process for the catheter 10B.

While the foregoing is directed to embodiments of the present invention, other and further embodiments of the invention may be devised without departing from the basic scope thereof, and the scope thereof is determined by the claims that follow.

What is claimed is:

1. A catheter, comprising:
a reinforced tube including a plurality of concentric bonded tubular layers of non-radiopaque material and a radiopaque, coil spring captured between adjacent ones of said tubular layers, one end of said reinforced tube providing an annular mounting portion and a tubular mounting member extending outwardly of said annular mounting portion; and
a tapered tip of radiopaque material including a tip central portion providing a tip central passageway receiving said tubular mounting member and a tip mounting portion abutting and bonded to said annular mounting portion, said tip central portion bonded to said tubular mounting member.

2. The catheter according to claim 1 wherein said tubular layers include an innermost tubular layer including an end extending outwardly of said annular mounting portion and providing said tubular mounting member.

3. The catheter according to claim 1 wherein said tubular layers include an innermost tubular layer including a first end extending outwardly of said tubular mounting member and a next adjacent tubular layer including a second end extending outwardly of said annular mounting portion, said first end and said second end combining to provide said tubular mounting member.

4. The catheter according to claim 3 wherein said tubular layers include an outermost tubular layer including an end providing said annular mounting portion and having a first durometer, wherein said next adjacent tubular layer has a second durometer softer than said first durometer, and wherein said tapered tip has a third durometer softer than said first durometer and harder than said second durometer.

5. The catheter according to claim 4 wherein said radiopaque, coil spring is a flat-wire radiopaque, coil spring having spaces between adjacent turns, wherein said flat-wire radiopaque, coil spring is captured between said outermost tubular layer and said next adjacent tubular layer, and wherein portions of said outermost tubular layer fill some of said spaces and wherein portions of said next adjacent tubular layer fill other of said spaces.

6. A catheter including a proximal catheter portion and a distal catheter portion, comprising:
a central tubular layer of low friction, non-radiopaque material extending through said proximal catheter portion and through said distal catheter portion and providing a catheter central passageway;
an inner tubular layer of non-radiopaque material surrounding and bonded to said central tubular layer of low friction, non-radiopaque material and extending through said proximal catheter portion and through said distal catheter portion to provide a distal portion;
an outer tubular layer of non-radiopaque material surrounding and bonded to said inner tubular layer of non-radiopaque material and extending only through said proximal catheter portion, said outer tubular layer of non-radiopaque material including a distal portion;
a radiopaque, coil spring captured between said inner tubular layer of non-radiopaque material and said outer tubular layer of non-radiopaque material and extending through at least a portion of said proximal catheter portion; and
a tapered tip of radiopaque material providing a tip central passageway receiving said distal portion of said inner tubular layer of non-radiopaque material, said tapered tip of radiopaque material bonded to said distal portion of said inner layer of non-radiopaque material and said tapered tip of radiopaque material including a proximal portion abutting and bonded to said distal end of said outer tubular layer of non-radiopaque material.

7. The claim according to claim 6 wherein said inner tubular layer of non-radiopaque material has a first durometer, wherein said outer tubular layer of non-radiopaque material has a second durometer harder than said first durometer and wherein said tapered tip of radiopaque material has a third durometer harder than said first durometer and softer than said second durometer.

8. The catheter according to claim 6 wherein said central tubular layer of low friction, non-radiopaque material comprises a tubular layer of low friction, non-radiopaque thermoplastic material providing lubricious conduit to medical instruments passing through said catheter central passageway.

9. The catheter according to claim 8 wherein said thermoplastic material is fluorinated ethylene propylene (FEP) or polytetrafluoroethylene (PTFE).

10. The catheter according to claim 6 wherein said inner tubular layer of non-radiopaque material is an inner tubular layer of non-radiopaque thermoplastic material having a durometer of about 20 to about 30 on the Shore D scale.

11. The catheter according to claim 10 wherein said thermoplastic material is a polyether block amide.

12. The catheter according to claim 6 wherein said outer tubular layer of non-radiopaque material is an outer tubular layer of non-radiopaque thermoplastic material having a durometer of about 50 to about 70 on the Shore D scale.

13. The catheter according to claim 12 wherein said thermoplastic material is a polyether block amide.

14. The catheter according to claim 6 wherein said tapered tip of radiopaque material is a tapered tip of thermoplastic material having a durometer of about 30 to about 45 on the Shore D scale and which is filled with about 70% to about 90% by weight of a radiopaque agent.

15. The catheter according to claim 14 wherein said thermoplastic material is a polyether block amide.

16. The catheter according to claim 14 wherein said radiopaque agent is tungsten.

17. A catheter including a catheter proximal portion and a catheter distal portion, comprising:
a plurality of at least three concentric, bonded tubular layers of non-radiopaque material, the innermost tubular layer also being low friction material;
the innermost tubular layer and the intermediate tubular layer extending through the catheter proximal portion and through the catheter distal portion to provide an intermediate tubular layer distal portion, the outermost tubular layer extending only through the catheter proximal portion and including a distal end;
a radiopaque, coil spring captured between the outermost tubular layer and the intermediate tubular layer in the catheter proximal portion; and
a tapered tip of radiopaque material including a tip proximal portion and a tip central portion providing a central passageway receiving said distal portion of said intermediate tubular layer, said tip central portion bonded to said distal portion of said intermediate tubular layer and said tip proximal portion bonded to said distal end of said outermost tubular layer.

18. The process of manufacturing a catheter, comprising the steps of:
providing a reinforced tube including a plurality of concentric bonded tubular layers of non-radiopaque material and a radiopaque, coil spring captured between adjacent ones of the tubular layers, providing an annular mounting portion at one end of the reinforced tube and providing a tubular mounting member extending outwardly of the annular mounting portion, and providing a tapered tip of radiopaque material including a tip central portion providing a tip central passageway and a tip annular mounting portion, bonding the tip annular mounting portion to the tube annular mounting portion and bonding the tip central portion to the tubular mounting member.

19. The process of manufacturing a catheter, comprising the steps of:
providing a central tubular layer of low friction, non-radiopaque material having a catheter central passageway;
surrounding said central tubular layer of low-friction, non-radiopaque material with an inner tubular layer of non-radiopaque material including a proximal portion and a distal portion;
surrounding at least a portion of said proximal portion of said inner tubular layer of non-radiopaque material with a radiopaque, coil spring having spaces between adjacent turns of said radiopaque, coil spring;
surrounding said radiopaque, coil spring and said inner tubular layer of non-radiopaque material with an outer tubular layer of non-radiopaque material including a proximal portion and a distal portion;
providing a tapered tip of radiopaque material including a tip central portion providing a tip central passageway and including a tip proximal portion and surrounding said distal portion of said inner tubular layer of non-radiopaque material with said tapered tip with said tip central passageway receiving said distal portion of said inner tubular layer of non-radiopaque material and with said tip proximal portion abutting said distal portion of said outer tubular layer of non-radiopaque material; and
bonding said central tubular layer of low-friction, non-radiopaque material to said inner tubular layer of non-radiopaque material, bonding said inner tubular layer of non-radiopaque material to said outer tubular layer of non-radiopaque material to capture said radiopaque, coil spring therebetween and to cause portions of said inner tubular layer of non-radiopaque material and portions of said outer tubular layer of non-radiopaque material to extend into said spaces between said adjacent turns of said radiopaque, coil spring, and bonding said tip proximal portion to said distal portion of said outer tubular layer of non-radiopaque material and bonding said tip central portion to said distal portion of said inner tubular layer of non-radiopaque material.

20. A catheter including a proximal catheter portion and a distal catheter portion, comprising:
a central tubular layer of low friction, non-radiopaque material extending through said proximal catheter portion and through said distal catheter portion to provide a distal portion and further providing a catheter central passageway;
an outer tubular layer of non-radiopaque material surrounding and bonded to said central tubular layer of low friction, non-radiopaque material and extending only through said proximal catheter portion, said outer tubular layer of non-radiopaque material including a distal end;
a radiopaque, coil spring captured between said central tubular layer of low friction, non-radiopaque material and said outer tubular layer of non-radiopaque material and extending through at least a portion of said proximal catheter portion; and
a tapered tip of radiopaque material providing a tip central passageway receiving said distal portion of said central tubular layer of low friction, non-radiopaque material, said tapered tip of radiopaque material bonded to said distal portion of said central layer of low friction, non-radiopaque material and said tapered tip of radiopaque material including a proximal portion abutting and bonded to said distal end of said outer tubular layer of non-radiopaque material.

21. The catheter according to claim 20 wherein said outer tubular layer of non-radiopaque material has a first durometer and wherein said tapered tip of radiopaque material has a second durometer softer than said first durometer.

22. The catheter according to claim 20 wherein said central tubular layer of low friction, non-radiopaque material comprises a tubular layer of low friction, non-radiopaque thermoplastic material providing lubricious conduit to medical instruments passing through said catheter central passageway.

23. The catheter according to claim 22 wherein said thermoplastic material is etched polytetrafluoroethylene (PTFE).

24. The catheter according to claim 20 wherein said outer tubular layer of non-radiopaque material is an outer tubular layer of non-radiopaque thermoplastic material having a durometer of about 50 to about 70 on the Shore D scale.

25. The catheter according to claim 24 wherein said thermoplastic material is a polyether block amide.

26. The catheter according to claim 20 wherein said tapered tip of radiopaque material is a tapered tip of thermoplastic material having a durometer of about 30 to about 45 on the Shore D scale and which is filled with about 70% to about 90% by weight of a rediopaque agent.

27. The catheter according to claim 26 wherein said thermoplastic material is a polyether block amide.

28. The catheter according to claim 26 wherein said radiopaque agent is tungsten.

29. A catheter including a proximal catheter portion and a catheter distal portion, comprising:
at least two concentric tubular layers of non-radiopaque material, the inner tubular layer also being low friction material and extending through the catheter proximal portion and through the catheter distal portion to provide a tubular distal portion, the outer tubular layer extending only through the catheter proximal portion and including a distal end;
a radiopaque, coil spring captured between the inner tubular and the outer tubular layer in the catheter proximal portion; and
a tapered tip of radiopaque material including a tip proximal portion and tip central portion providing a central passageway receiving said tubular distal portion, said tip central portion bonded to said tubular distal portion and said tip proximal portion bonded to said distal end.

30. The process of manufacturing a catheter, comprising the steps of:
providing a central tubular layer of low friction, non-radiopaque material including a proximal portion and a distal portion and providing a catheter central passageway;
surrounding at least a portion of said proximal portion of said central tubular layer of low friction, non-radiopaque material with a radiopaque, coil spring having spaces between adjacent turns of said radiopaque, coil spring;
surrounding said radiopaque, coil spring and only said proximal portion of said central tubular layer of low friction, non-radiopaque material with an outer tubular layer of non-radiopaque material including a distal end;
providing a tapered tip of radiopaque material including a tip central portion providing a tip central passageway and further including a tip proximal portion and surrounding said distal portion of said central tubular layer of low friction, non-radiopaque material with said tapered tip with said tip central passageway receiving said distal portion of said central tubular layer of low friction, non-radiopaque material and with said tip proximal portion abutting said distal end of said outer tubular layer of non-radiopaque material; and
bonding said central tubular layer of low-friction, non-radiopaque material to said outer tubular layer of non-radiopaque material to capture said radiopaque, coil spring therebetween and to cause portions of said outer tubular layer of non-radiopaque material to extend into said spaces between said adjacent turns of said radiopaque, coil spring, and bonding said tip proximal portion to said distal end of said outer tubular layer of non-radiopaque material and bonding said tip central portion to said distal portion of said central tubular layer of low friction, non-radiopaque material.
